SEQUENCE LISTING (38 sequences)

SEQ ID NO: 1 - protein, 245 aa, Corynebacterium glutamicum
  REGION 1..245: note = BBD29_13140
MNQDLSHEDSGDGNSVDRGQILLAVLIGLALIASVIMLLANSDGAMKIALLAALWAAIIGFFLVYRSRKQVEAAAREKETLEYAHQSELNRLEAELVQEKMEISESRRARDQETLEEIKLQLEEMRTQLSELSGREWGYEPTMLRAEARRILELESQQLSQQFQAPQPEVPEPVAVPEPTPEPAPVPEPEPVEVAVEAEEEPAPGRRRRRHAAPEEAGGRRRKDERQGGLSVADLLAAARKKENN

SEQ ID NO: 2 - DNA, 738 bp, Corynebacterium glutamicum
  misc_feature 1..738: note = BBD29_13140
  source 1..738: mol_type = genomic DNA
atgaatcaagacctgtcacacgaggattccggcgacggcaattctgtcgaccgtggacagattctgctagcagtactcattggacttgccctgattgccagcgtcatcatgctgctggccaacagtgacggcgccatgaaaatagcacttcttgcagcgctgtgggcggcaatcatcggattcttccttgtttatcggtcccgcaaacaagtcgaagctgctgcgcgggagaaggaaaccctcgaatacgcacaccaatctgaactcaaccgactagaagctgaactcgtccaagaaaaaatggagatttctgaatcccgtcgtgcacgcgatcaggaaacccttgaggaaatcaaacttcaactggaggaaatgcgcacccagctgtctgagctttctggccgtgaatggggctatgagccaaccatgctgcgtgccgaagcccgacgaatccttgagttggaatcccagcagctttcccagcagttccaggcaccgcagccagaagtcccggagcctgttgcagttccagagccaacgccggaacctgcaccggtgccggaacctgagccagttgaagttgctgtcgaagctgaggaagaaccagcaccaggtcgcagaaggcgtaggcacgcggctcccgaagaagctggcggacgccgacgcaaagatgaacgccaaggcggtttgagcgtggctgatcttctggcagcagcaaggaaaaaggaaaacaactaa

SEQ ID NO: 3 - DNA, 38 bp, synthetic construct
  misc_feature 1..38: note = 1F
  source 1..38: mol_type = other DNA
ttcgagctcggtaccccgatctactcaaccccaccgtg

SEQ ID NO: 4 - DNA, 38 bp, synthetic construct
  misc_feature 1..38: note = 2R
  source 1..38: mol_type = other DNA
acaggtcttgattcacgaattccaatctagctgatcgc

SEQ ID NO: 5 - DNA, 38 bp, synthetic construct
  misc_feature 1..38: note = 3F
  source 1..38: mol_type = other DNA
agctagattggaattcgtgaatcaagacctgtcacacg

SEQ ID NO: 6 - DNA, 34 bp, synthetic construct
  misc_feature 1..34: note = 4R
  source 1..34: mol_type = other DNA
actctagaggatccccgatcagccacgctcaaac

SEQ ID NO: 7 - DNA, 38 bp, synthetic construct
  misc_feature 1..38: note = 5R
  source 1..38: mol_type = other DNA
acaggtcttgattcaagaattccaatctagctgatcgc

SEQ ID NO: 8 - DNA, 38 bp, synthetic construct
  misc_feature 1..38: note = 6F
  source 1..38: mol_type = other DNA
agctagattggaattcttgaatcaagacctgtcacacg

SEQ ID NO: 9 - DNA, 38 bp, synthetic construct
  misc_feature 1..38: note = 7F
  source 1..38: mol_type = other DNA
tgaattcgagctcggtacccttgaacggaattgggtgg

SEQ ID NO: 10 - DNA, 38 bp, synthetic construct
  misc_feature 1..38: note = 8R
  source 1..38: mol_type = other DNA
cccaggtggcatcggtaccttcacccagcgccacgcag

SEQ ID NO: 11 - DNA, 39 bp, synthetic construct
  misc_feature 1..39: note = 9F
  source 1..39: mol_type = other DNA
cgctgggtgaaggtaccgatgccacctgggttggtcaag

SEQ ID NO: 12 - DNA, 38 bp, synthetic construct
  misc_feature 1..38: note = 10R
  source 1..38: mol_type = other DNA
gtcgactctagaggatccccggacaaggaatggagaga

SEQ ID NO: 13 - DNA, 18 bp, synthetic construct
  misc_feature 1..18: note = 11F
  source 1..18: mol_type = other DNA
cttaccgttgttgccctt

SEQ ID NO: 14 - DNA, 19 bp, synthetic construct
  misc_feature 1..19: note = 12R
  source 1..19: mol_type = other DNA
ctccttcacccacatcatt

SEQ ID NO: 15 - DNA, 16 bp, synthetic construct
  misc_feature 1..16: note = 13F
  source 1..16: mol_type = other DNA
gatcgctacgcgctgc

SEQ ID NO: 16 - DNA, 18 bp, synthetic construct
  misc_feature 1..18: note = 14R
  source 1..18: mol_type = other DNA
gcgaggagcctgcattag

SEQ ID NO: 17 - DNA, 38 bp, synthetic construct
  misc_feature 1..38: note = 15F
  source 1..38: mol_type = other DNA
tgaattcgagctcggtaccccgcctttgtgttgatccc

SEQ ID NO: 18 - DNA, 48 bp, synthetic construct
  misc_feature 1..48: note = 16R
  source 1..48: mol_type = other DNA
ggtcttgattcatgaattccggccttgctgatcgcttcgccagattgg

SEQ ID NO: 19 - DNA, 48 bp, synthetic construct
  misc_feature 1..48: note = 17F
  source 1..48: mol_type = other DNA
ccaatctggcgaagcgatcagcaaggccggaattcatgaatcaagacc

SEQ ID NO: 20 - DNA, 38 bp, synthetic construct
  misc_feature 1..38: note = 18R
  source 1..38: mol_type = other DNA
gtcgactctagaggatccccgctctggaactgcaacag

SEQ ID NO: 21 - DNA, 48 bp, synthetic construct
  misc_feature 1..48: note = 19R
  source 1..48: mol_type = other DNA
ggtcttgattcatgaattcctgtcacgctgatcgcttcgccagattgg

SEQ ID NO: 22 - DNA, 48 bp, synthetic construct
  misc_feature 1..48: note = 20F
  source 1..48: mol_type = other DNA
ccaatctggcgaagcgatcagcgtgacaggaattcatgaatcaagacc

SEQ ID NO: 23 - DNA, 48 bp, synthetic construct
  misc_feature 1..48: note = 21R
  source 1..48: mol_type = other DNA
ggtcttgattcatgaattccagcgaagctgatcgcttcgccagattgg

SEQ ID NO: 24 - DNA, 48 bp, synthetic construct
  misc_feature 1..48: note = 22F
  source 1..48: mol_type = other DNA
ccaatctggcgaagcgatcagcttcgctggaattcatgaatcaagacc

SEQ ID NO: 25 - DNA, 48 bp, synthetic construct
  misc_feature 1..48: note = 23R
  source 1..48: mol_type = other DNA
ggtcttgattcatgaattgaaatcgacctgatcgcttcgccagattgg

SEQ ID NO: 26 - DNA, 48 bp, synthetic construct
  misc_feature 1..48: note = 24F
  source 1..48: mol_type = other DNA
ccaatctggcgaagcgatcaggtcgatttcaattcatgaatcaagacc

SEQ ID NO: 27 - DNA, 17 bp, synthetic construct
  misc_feature 1..17: note = 25F
  source 1..17: mol_type = other DNA
cacgcaccctcgatgtg

SEQ ID NO: 28 - DNA, 17 bp, synthetic construct
  misc_feature 1..17: note = 26R
  source 1..17: mol_type = other DNA
ccagcttcttcgggagc

SEQ ID NO: 29 - DNA, 35 bp, synthetic construct
  misc_feature 1..35: note = 27R
  source 1..35: mol_type = other DNA
gaaggcgaggagcctgcagaattccaatctagctg

SEQ ID NO: 30 - DNA, 35 bp, synthetic construct
  misc_feature 1..35: note = 28F
  source 1..35: mol_type = other DNA
cagctagattggaattctgcaggctcctcgccttc

SEQ ID NO: 31 - DNA, 35 bp, synthetic construct
  misc_feature 1..35: note = 29R
  source 1..35: mol_type = other DNA
actctagaggatcccccagctcaatatcttgcgcg

SEQ ID NO: 32 - DNA, 17 bp, synthetic construct
  misc_feature 1..17: note = 30F
  source 1..17: mol_type = other DNA
cccaaatgaaccccgtg

SEQ ID NO: 33 - DNA, 17 bp, synthetic construct
  misc_feature 1..17: note = 31R
  source 1..17: mol_type = other DNA
gatgccgtgcgtgagcg

SEQ ID NO: 34-38 - skipped (5 entries)

(19) United States
(12) Patent Application Publication
(10) Pub. No.: US 2024/0401066 A1
(43) Pub. Date: Dec. 5, 2024

(54) STRAIN FOR PRODUCING HIGH CONCENTRATION L-GLUTAMIC ACID AND METHOD FOR PRODUCING L-GLUTAMIC ACID USING THE SAME

(71) Applicant: CJ CHEILJEDANG CORPORATION, Seoul (KR)

(72) Inventors: Nara KWON, Seoul (KR); Hyun-Ju BONG, Seoul (KR); Ah Reum LEE, Seoul (KR); Jung Ok HEO, Seoul (KR)

(73) Assignee: CJ CHEILJEDANG CORPORATION, Seoul (KR)

(21) Appl. No.: 18/694,893

(22) PCT Filed: Sep. 22, 2022
(86) PCT No.: PCT/KR2022/014177

§ 371 Date: Mar. 22, 2024

(30) Foreign Application Priority Data
Sep. 23, 2021 (KR) 10-2021-0125842

(51) Int. Cl. C12N 15/77; C07K 14/34; C12P 13/18

(52) U.S. Cl. CPC C12N 15/77; C07K 14/34; C12P 13/18

(57) ABSTRACT

The present application relates to a strain and a microorganism for producing high concentration L-glutamic acid and a method of use thereof.

### TECHNICAL FIELD

The present disclosure relates to a strain for producing high concentration L-glutamic acid and a method for producing L-glutamic acid using the same.

### BACKGROUND ART

L-glutamic acid is a representative amino acid produced by fermentation, and is one of the important amino acids widely used not only in the food field but also in the pharmaceutical field, other animal feed fields, etc. because of its unique taste. Methods for producing L-glutamic acid using microorganisms such as the genus Corynebacterium, Escherichia coli, Bacillus, Streptomyces, the genus Penicillum, Klebsiella, Erwinia, and the genus Pantoea are known (U.S. Pat. Nos. 3,220,929 and 6,682,912).

Currently, various studies are being conducted to develop microorganisms and fermentation process technology that produce L-glutamic acid at a high efficiency. For example, a target substance-specific approach such as increasing the expression of a gene encoding an enzyme involved in amino acid biosynthesis or removing a gene unnecessary for amino acid biosynthesis in a microorganism is mainly utilized to improve the production yield of L-glutamic acid.

### DETAILED DESCRIPTION

One object of the present application is to provide a microorganism, in which an activity of a protein comprising a sequence having at least 85% sequence identity to the amino acid sequence of SEQ ID NO: 1 is weakened compared to an intrinsic activity of the microorganism. Another object of the present application is to provide a microorganism expressing a recombinant protein comprising an amino acid sequence having at least 85% sequence identity to the amino acid sequence of SEQ ID NO: 1.

Another object of the present disclosure is to provide a method for producing L-glutamic acid, comprising culturing the microorganism of the present disclosure in a medium.

Still another object of the present disclosure is to provide a composition for L-glutamic acid production, comprising the microorganism of the present disclosure; a medium in which the microorganism has been cultured; or a combination thereof.

Still another object of the present disclosure is to provide a method for producing a microorganism, comprising weakening the activity of the protein of the present disclosure. In some embodiments, the microorganism is Corynebacterium.

Yet still another object of the present disclosure is to provide a method of increasing L-glutamic acid production by a microorganism, comprising weakening an activity of a protein comprising an amino acid sequence having at least 85% sequence identity to the amino acid sequence of SEQ ID NO: 1 in the microorganism.

Each description and embodiment disclosed in the present disclosure may also be applied to other descriptions and embodiments. That is, all combinations of various elements disclosed in the present disclosure fall within the scope of the present application. Further, the scope of the present application is not limited by the specific description below. In addition, those skilled in the art will recognize or be able to ascertain using no more than routine experimentation, many equivalents to the specific aspects of the present application described herein. Further, these equivalents should be interpreted to fall within the scope of the present application.

One aspect of the present disclosure is to provide a microorganism, in which the activity of the protein comprising an amino acid sequence having at least 70% sequence identity to SEQ ID NO: 1 is weakened. Another aspect of the present disclosure is to provide a microorganism expressing a recombinant protein comprising an amino acid sequence having at least 70% sequence identity to the amino acid sequence of SEQ ID NO: 1. In some embodiments, the microorganism is of the genus Corynebacterium.

The protein according to some embodiments of the present disclosure above may have, include, or consist of the amino acid sequence set forth in SEQ ID NO: 1, or essentially consist of the amino acid sequence. Specifically, the protein according to some embodiments of the present disclosure may consist of a polypeptide set forth in the amino acid sequence of SEQ ID NO: 1.

The amino acid sequence of SEQ ID NO: 1 may be obtained from National Institutes of Health (NIH) GenBank, a known database. In the present disclosure, the amino acid sequence of SEQ ID NO: 1 may include an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 9%, 93%, 94%, 95%, 95.18%, 96%, 97%, 98%, 99%, 99.5%, 99.7%, or 99.9% of homology or identity with the amino acid sequence set forth in SEQ ID NO: 1. In some embodiments, the protein of the microorganism described above may exclude SEQ ID NO: 1. In addition, it is apparent that a protein having an amino acid sequence in which some sequences are deleted, modified, substituted, conservatively substituted or added is also included within the scope of the present disclosure as long as the amino acid sequence has such homology or identity and exhibits efficacy corresponding to the protein including the amino acid sequence of SEQ ID NO: 1.

Examples thereof include cases having sequence additions or deletions, naturally occurring mutations, silent mutations or conservative substitutions that do not alter the function of the protein according to some embodiments of the present disclosure at the N-terminus, C-terminus and/or within the amino acid sequence.

The term “conservative substitution” means substituting an amino acid with another amino acid having similar structural and/or chemical properties. Such amino acid substitutions may generally occur based on similarity in polarity, charge, solubility, hydrophobicity, hydrophilicity and/or amphipathic nature of the residues. Typically, conservative substitutions may have little or no effect on the activity of the protein or polypeptide.

As used herein, the term “homology” or “identity” refers to the degree of similarity between two given amino acid sequences or nucleotide sequences and may be expressed as a percentage. The terms homology and identity may often be used interchangeably.

The sequence homology or identity of a conserved polynucleotide or polypeptide is determined by standard alignment algorithms, and a default gap penalty established by the program being used may be used together. Substantially homologous or identical sequences are generally capable of hybridizing with all or part of the sequence under moderate or high stringent conditions. It is apparent that hybridization also includes hybridization with a polynucleotide containing a common codon in a polynucleotide or a codon taking codon degeneracy into account.

Whether or not any two polynucleotide or polypeptide sequences have homology, similarity, or identity may be determined, for example, using known computer algorithms such as the “FASTA” program using default parameters as in Pearson et al. (1988) [Proc. Natl. Acad. Sci. USA 85]: 2444. Alternatively, the homology, similarity or identity may be determined using the Needleman-Wunsch algorithm (Needleman and Wunsch, 1970, J. Mol. Biol. 48:443-453) as performed in the Needleman program of the EMBOSS package (EMBOSS: The European Molecular Biology Open Software Suite, Rice et al., 2000, Trends Genet. 16:276-277) (version 5.0.0 or later) (including GCG program package (Devereux, J., et al., Nucleic Acids Research 12:387 (1984), BLASTP, BLASTN, FASTA (Atschul, [S.] [F.,] [et al., J. Mol. Biol. 215]: 403 (1990): Guide to Huge Computers, Martin J. Bishop, [ED.,] Academic Press, San Diego, 1994, and [CARILLO et al.] (1988) SIAM J Applied Math 48:1073). For example, BLAST of the National Center for Biotechnology Information, or ClustalW may be used to determine the homology, similarity, or identity.

The homology, similarity or identity of polynucleotides or polypeptides may be determined by comparing sequence information, for example, using a GAP computer program such as Needleman et al. (1970), J Mol Biol. 48:443, for example, as known in Smith and Waterman, Adv. Appl. Math. (1981) 2:482. In summary, a GAP program may be defined as the value acquired by dividing the total number of symbols in the shorter of two sequences by the number of similarly aligned symbols (namely, nucleotides or amino acids). Default parameters for the GAP program may include (1) binary comparison matrix (containing values of 1 for identity and 0 for non-identity) and weighted comparison matrix of Gribskov et al., (1986) Nucl. Acids Res. 14:6745 as disclosed in Schwartz and Dayhoff, eds., Atlas Of Protein Sequence And Structure, National Biomedical Research Foundation, pp. 353-358 (1979) (or EDNAFULL (EMBOSS version of NCBI NUC4.4) substitution matrix): (2) a penalty of 3.0 for each gap and an additional 0.10 penalty for each symbol in each gap (or a gap opening penalty of 10, a gap extension penalty of 0.5); and (3) no penalty for an end gap.

The protein according to some embodiments of the present disclosure may be derived from a microorganism. In some embodiments, the microorganism may be Corynebacterium, Escherichia coli, Bacillus, Streptomyces, Penicillum, Klebsiella, Erwinia, or Pantoea. The microorganism may be specifically derived from a microorganism of the genus Corynebacterium, more specifically derived from Corynebacterium glutamicum, Corynebacterium deserti, Corynebacterium crenatum, Corynebacterium efficiens, Corynebacterium suranareeae, and the like, but is not limited thereto.

The protein that is inactivated in the microorganism provided in the present disclosure is represented by SEQ ID NO: 1, but sequences having a similar structure and exhibiting similar activity thereto may be included without limitation.

For example, the protein inactivated in the microorganism of the present disclosure includes a protein derived from the genus Corynebacterium having 70% or more identity with SEQ ID NO: 1. Specifically, the protein may be a protein derived from Corynebacterium glutamicum having 85, 86, 87, 88, 89, 90, 91, 92, 93, 94, 95, 96, 97, 98, or 99% or more identity with SEQ ID NO: 1. Examples thereof include proteins such as NCBI Accession No. WP_060565206.1, QWQ85246.1, WP_065367112.1, WP_038585884.1, WP_040072989.1, WP_011265991.1, WP_074492394.1, ARV66101.1, WP_003853812.1, WP_006285921.1, WP 003862956.1, WP_044027349.1, BAF55605.1, WP_179205783.1, WP_172769145.1, WP_211439424.1, WP_179111498.1, WP_059289862.1, WP_185969420.1, WP_173673681.1, and QDQ21801.1, but are not limited thereto.

The polynucleotide encoding the protein according to some embodiments of the present disclosure may include a nucleotide sequence encoding the amino acid sequence set forth in SEQ ID NO: 1. The polynucleotide according to some embodiments of the present disclosure may be, for example, a polynucleotide sequence having a locus tag, such as FOL53_14395. B7P23_15040. KaCgl_12360. B5C28_13285, cgR_2591, or BBD29_13140, derived from the genus Corynebacterium, and examples thereof may include BBD29_13140 derived from Corynebacterium glutamicum ATCC13869, but are not limited thereto. The BBD29_13140 may have, include, or consist of the nucleotide sequence set forth in SEQ ID NO: 2, or may essentially consist of the nucleotide sequence.

As used herein, the term “polynucleotide” refers to a DNA or RNA strand of a certain length or longer as a polymer of nucleotides in which nucleotide monomers are linked in a long chain shape by covalent bonds, more specifically a polynucleotide fragment encoding the protein.

In the polynucleotide according to some embodiments of the present disclosure, various modifications may be made in the coding region within a range in which the amino acid sequence of the protein is not changed in consideration of codon degeneracy or preferred codons in organisms to express the protein described herein.

Another aspect of the present disclosure is to provide a polynucleotide comprising a nucleic acid sequence encoding the protein described above, wherein the polynucleotide does not occur in nature.

In some embodiments, the polynucleotide of the present disclosure has or includes a nucleotide sequence having 70, 71, 72, 73, 74, 75, 76, 77, 78, 79, 80, 81, 82, 83, 84, 85, 86, 87, 88, 89, 90, 91, 92, 93, 94, 95, 96, 97, or 98% or more homology or identity with the sequence of SEQ ID NO: 2, or may consist of or essentially consist of a nucleotide sequence having 70, 71, 72, 73, 74, 75, 76, 77, 78, 79, 80, 81, 82, 83, 84, 85, 86, 87, 88, 89, 90, 91, 92, 93, 94, 95, 96, 97, or 98% or more homology or identity with the sequence of SEQ ID NO: 2, but is not limited thereto. In some embodiments, the nucleic acid sequence comprises a start codon that does not naturally occur in encoding the amino acid sequence. In some embodiments, an intrinsic gene encoding the amino acid sequence comprises a start codon of ATG, and the ATG is replaced with GTG. In some embodiments, an intrinsic gene encoding the amino acid sequence comprises a start codon of ATG, and the ATG is replaced with TTG. In some embodiments, the nucleic acid sequence comprises a Shine-Dalgarno sequence that does not naturally occur in encoding the amino acid sequence. In some embodiments, an intrinsic gene encoding the amino acid sequence comprises a Shine-Dalgarno sequence of CTAGATTGG, and the CTAGATTGG is replaced with CAAGGCCGG. In some embodiments, an intrinsic gene encoding the amino acid sequence comprises a Shine-Dalgarno sequence of CTAGATTGG, and the CTAGATTGG is replaced with CGTGACAGG. In some embodiments, an intrinsic gene encoding the amino acid sequence comprises a Shine-Dalgarno sequence of CTAGATTGG, and the CTAGATTGG is replaced with CTTCGCTGG. In some embodiments, an intrinsic gene encoding the amino acid sequence comprises a Shine-Dalgarno sequence of CTAGATTGG, and the CTAGATTGG is replaced with GTCGATTTC.

In addition, the polynucleotide according to some embodiments of the present disclosure may include any probe that can be prepared from a known gene sequence, for example, any sequence that can hybridize with a sequence complementary to all or part of the polynucleotide sequence of the present disclosure under stringent conditions without limitation. The “stringent condition” refers to a condition that enables specific hybridization between polynucleotides. Such conditions are specifically described in the literatures (see J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York, 9.50-9.51, 11.7-11.8). Examples thereof include a condition in which polynucleotides having high homology or identity, namely polynucleotides having 70% or more, 75% or more, 76% or more, 85% or more, 90% or more, 95% or more, 96% or more, 97% or more, 98% or more, or 99% or more homology or identity hybridize with each other and polynucleotides having homology or identity lower than this do not hybridize with each other; or a condition in which washing is performed one time, specifically 2 to 3 times at a salt concentration and a temperature equivalent to 60° C., 1×SSC, 0.1% SDS, specifically 60° C., 0.1×SSC, 0.1% SDS, more specifically 68° C., 0.1×SSC, 0.1% SDS, which are the washing conditions of ordinary southern hybridization.

Hybridization requires that two nucleic acids have complementary sequences although mismatch between bases is possible depending on the stringency of hybridization. The term “complementary” is used to describe the relation between nucleotide bases capable of hybridizing with each other. For example, with regard to DNA, adenine is complementary to thymine and cytosine is complementary to guanine. Accordingly, the polynucleotide described herein may also include substantially similar nucleic acid sequences as well as isolated nucleic acid fragments complementary to the complete sequence.

Specifically, a polynucleotide having homology or identity with the polynucleotide described herein may be detected using hybridization conditions including a hybridization step at a Tm value of 55° C. and the conditions described above. In addition, the Tm value may be 60° C., 63° C., or 65° C., but is not limited thereto and may be appropriately adjusted by those skilled in the art depending on the purpose.

The appropriate stringency for hybridizing polynucleotides depends on the length and the degree of complementarity of the polynucleotides, and the parameters are well known in the art (for example, J. Sambrook et al., supra).

As used herein, the term “microorganism (or strain)” includes both wild-type microorganisms or microorganisms in which genetic modification has occurred naturally or artificially, and may refer to a microorganism in which a specific mechanism is weakened or enhanced by causes such as insertion of an exogenous gene or enhancement or inactivation of the activity of an endogenous gene, containing genetic modification to produce a desired polypeptide, protein, or product.

As used herein, the term “weakening” of the activity of a polypeptide is a concept in which the activity is decreased compared to the intrinsic activity, and the weakening may be used interchangeably with terms such as down-regulation, decrease, reduction, and attenuation.

Meanwhile, in the present disclosure, the weakening of protein activity is a state in which the protein exhibits activity but is not completely inactivated by deletion, and may mean a case in which the protein exhibits lower activity compared to the wild-type strain or parent strain.

The weakening may include a case in which the activity of the polypeptide itself is decreased compared to the original activity of the polypeptide possessed by the microorganism by mutation of the polynucleotide encoding the polypeptide, and the like, and a case in which the overall polypeptide activity degree and/or concentration (expression level) in the cell is lower than that in the native strain because of inhibition of the expression of the gene of the polynucleotide encoding the polypeptide or inhibition of translation into the polypeptide. The “intrinsic activity” refers to the activity of a specific polypeptide originally possessed by the parent strain before transformation or the wild-type microorganism or unmodified microorganism when the trait is changed by genetic mutation due to natural or artificial factors. The intrinsic activity may be used interchangeably with “activity before modification”. When the activity of a polypeptide is “weakened, decreased, down-regulated, reduced, or attenuated” compared to the intrinsic activity, it means that the activity of a polypeptide is lowered compared to the activity of a specific polypeptide originally possessed by the parent strain before transformation or unmodified microorganism.

Such weakening of the activity of a polypeptide may be performed by any method known in the art, but is not limited thereto, and may be achieved by applying various methods well known in the art (for example, Nakashima N et al., Bacterial cellular engineering by genome editing and gene silencing. Int J Mol Sci. 2014: 15 (2): 2773-2793, Sambrook et al. Molecular Cloning 2012).

In some embodiments, the weakening of the activity of a polypeptide of the present disclosure may be achieved by:
1) deletion of part of a gene encoding a polypeptide:
2) modification of the expression control region (or expression control sequence) to decrease the expression of a gene encoding a polypeptide;
3) modification of the amino acid sequence constituting the polypeptide to weaken the activity of the polypeptide (for example, deletion/substitution/addition of one or more amino acids on the amino acid sequence);
4) modification of the gene sequence encoding a polypeptide to weaken the activity of the polypeptide (for example, deletion/substitution/addition of one or more nucleotides on the nucleotide sequence of the polypeptide gene to encode a polypeptide that has been modified to weaken the activity of the polypeptide);
5) modification of the nucleotide sequence encoding the start codon, Shine-Dalgarno sequence or 5′-UTR region of a gene transcript encoding a polypeptide;
6) introduction of an antisense oligonucleotide (for example, antisense RNA) that complementarily binds to the transcript of a gene encoding a polypeptide;
7) addition of a sequence complementary to the Shine-Dalgarno sequence in a front end of the Shine-Dalgarno sequence of a gene encoding a polypeptide to form a secondary structure to which a ribosome cannot be attached;
8) addition of a promoter transcribed in the opposite direction to the 3′ end of the open reading frame (ORF) of a gene sequence encoding a polypeptide (reverse transcription engineering, RTE): or
9) a combination of two or more selected from 1) to 8) above, but is not particularly limited thereto.

In the microorganism according to some embodiments of the present disclosure, at least a part of a nucleotide sequence coding the protein is deleted compared to an intrinsic nucleotide sequence of the microorganism. In some embodiments, a nucleotide sequence coding the protein is deleted.

In the microorganism according to additional embodiments of the present disclosure, an expression control sequence for a gene encoding the protein is not an intrinsic sequence of the microorganism. In some embodiments, the expression control sequence comprises at least one deletion, insertion or substitution in the intrinsic expression control sequence to reduce an expression of the gene encoding the protein. In some embodiments, the expression control sequence comprises at least one sequence selected from the group consisting of a promoter sequence, operator sequence, a sequence coding a ribosome binding site, a sequence coding termination of transcription, and a sequence coding termination of translation. As used herein, “expression control sequence” means a nucleic acid sequence that directs transcription of a nucleic acid. The expression control sequence may be a polynucleotide sequence that regulates expression of a polypeptide coded for by a polynucleotide to which it is functionally (“operably”) linked.

In the microorganism according to additional embodiments of the present disclosure, the amino acid sequence is not an intrinsic amino acid sequence of the microorganism. In some embodiments, the amino acid sequence comprises at least one deletion, insertion or substitution in the intrinsic amino acid sequence to weaken the activity of the protein.

In the microorganism according to additional embodiments of the present disclosure, a nucleotide sequence coding the protein is not an intrinsic nucleotide sequence of the microorganism. In some embodiments, the nucleotide sequence comprises at least one deletion, insertion or substitution in the intrinsic nucleotide sequence to weaken the activity of the protein.

In the microorganism according to additional embodiments of the present disclosure, at least one sequence selected from the group consisting of a nucleotide sequence for a start codon, a nucleotide sequence for stop codon, Shine-Dalgarno sequence, and 5′-UTR region of a gene encoding the protein is not an intrinsic sequence of the microorganism. In some embodiments, the nucleotide sequence for the start codon of the gene encoding the protein is mutated compared to the intrinsic sequence of the microorganism. In some embodiments, the nucleotide sequence for a stop codon of the gene encoding the protein is mutated compared to the intrinsic sequence of the microorganism. In some embodiments, an intrinsic gene encoding the protein comprises a start codon of ATG, and the ATG is replaced with GTG. In some embodiments, an intrinsic gene encoding the protein comprises a start codon of ATG, and the ATG is replaced with TTG. In some embodiments, the nucleotide sequence for a Shine-Dalgarno sequence of the gene encoding the protein is mutated compared to the intrinsic sequence of the microorganism. In some embodiments, an intrinsic gene encoding the protein comprises a Shine-Dalgarno sequence of CTAGATTGG, and the CTAGATTGG is replaced with CAAGGCCGG. In some embodiments, an intrinsic gene encoding the protein comprises a Shine-Dalgarno sequence of CTAGATTGG, and the CTAGATTGG is replaced with CGTGACAGG. In some embodiments, an intrinsic gene encoding the protein comprises a Shine-Dalgarno sequence of CTAGATTGG, and the CTAGATTGG is replaced with CTTCGCTGG. In some embodiments, an intrinsic gene encoding the protein comprises a Shine-Dalgarno sequence of CTAGATTGG, and the CTAGATTGG is replaced with GTCGATTTC.

In the microorganism according to additional embodiments of the present disclosure, the microorganism comprises an antisense oligonucleotide that complementarily binds to the transcript of a gene encoding the protein. In some embodiments, the antisense oligonucleotide comprises an antisense RNA.

In the microorganism according to additional embodiments of the present disclosure, a sequence complementary to a Shine-Dalgarno sequence of a gene encoding the protein is inserted in front of the Shine-Dalgarno sequence to form a secondary structure. In some embodiments, the secondary structure does not bind to the ribosome to reduce or delay mRNA translation.

In the microorganism according to additional embodiments of the present disclosure, a promoter for reverse transcription engineering (RTE) is inserted at 3′ end of open reading frame (ORF) of a gene encoding the protein. In some embodiments, an antisense nucleotide molecule complementary to at least a part of a gene encoding the protein is produced, thereby weakening the activity of a protein.

For example,
1) the deletion of part of a gene encoding a polypeptide may be replacement with a polynucleotide in which some nucleotides are deleted from a polynucleotide encoding an endogenous target polypeptide in a chromosome or replacement with a marker gene.
2) The modification of the expression control region (or expression control sequence) may be occurrence of mutation in the expression control region (or expression control sequence) by deletion, insertion, non-conservative or conservative substitution, or a combination thereof; or replacement with a sequence having weaker activity. The expression control region includes, but is not limited to, a promoter, an operator sequence, a sequence encoding a ribosome binding site, and a sequence regulating the termination of transcription and translation.
3) and 4) The modification of amino acid sequence or polynucleotide sequence may be, but is not limited to, occurrence of mutation in the amino acid sequence of a polypeptide or a polynucleotide sequence encoding the polypeptide by deletion, insertion, non-conservative or conservative substitution, or a combination thereof so that the activity of the polypeptide is weakened, or replacement of the sequence with an amino acid sequence or polynucleotide sequence modified to have weaker activity or an amino acid sequence or polynucleotide sequence modified to have no activity. For example, the expression of a gene may be inhibited or weakened by introducing a mutation into the polynucleotide sequence to form a stop codon, but the modification is not limited thereto.
5) The modification of the nucleotide sequence encoding the start codon or 5′-UTR region of a gene transcript encoding a polypeptide may be, for example, but is not limited to, substitution with a nucleotide sequence encoding another start codon having a lower expression rate of the polypeptide compared to the endogenous start codon.
For 6) the introduction of an antisense oligonucleotide (for example, antisense RNA) that complementarily binds to the transcript of the gene encoding a polypeptide, for example reference may be made to the literature [Weintraub, H. et al., Antisense-RNA as a molecular tool for genetic analysis, Reviews—Trends in Genetics, Vol. 1 (1) 1986].
7) The addition of a sequence complementary to the Shine-Dalgarno sequence in the front end of the Shine-Dalgarno sequence of a gene encoding a polypeptide to form a secondary structure to which a ribosome cannot be attached may be to disable or slow down mRNA translation.
8) The addition of a promoter transcribed in the opposite direction to the 3′ end of the open reading frame (ORF) of a gene sequence encoding a polypeptide (reverse transcription engineering, RTE) may be to create an antisense nucleotide complementary to the transcript of the gene encoding the polypeptide to weaken the activity.

As used herein, the term “enhancement” of polypeptide activity means that the activity of a polypeptide is increased compared to the intrinsic activity. The enhancement may be used interchangeably with terms such as activation, up-regulation, overexpression, and increase. Here, activation, enhancement, up-regulation, overexpression, and increase may include both exhibiting activity that is not originally possessed and exhibiting activity improved compared to the intrinsic activity or activity before modification. The “intrinsic activity” refers to the activity of a specific polypeptide originally possessed by the parent strain before transformation or the unmodified microorganism when the trait is changed by genetic mutation due to natural or artificial factors. The intrinsic activity may be used interchangeably with “activity before modification”. When the activity of a polypeptide is “enhanced, up-regulated, overexpressed, or increased” compared to the intrinsic activity, it means that the activity of a polypeptide is improved compared to the activity and/or concentration (expression level) of a specific polypeptide originally possessed by the parent strain before transformation or unmodified microorganism.

The enhancement may be achieved by introducing an exogenous polypeptide or by enhancing the activity and/or concentration (expression level) of the endogenous polypeptide. Whether or not the activity of a polypeptide is enhanced may be confirmed from an increase in the activity degree or expression level of the polypeptide or the amount of product excreted from the polypeptide.

For the enhancement of the activity of a polypeptide, various methods well known in the art may be applied, and the method is not limited as long as it can enhance the activity of a target polypeptide compared to that in the microorganism before modification. Specifically, the enhancement may be enhancement using genetic engineering and/or protein engineering well known to those skilled in the art, which is a routine method in molecular biology, but is not limited thereto (for example, Sitnicka et al., Functional Analysis of Genes. Advances in Cell Biology. 2010, Vol. 2. 1-16, Sambrook et al. Molecular Cloning 2012).

In some embodiments, the enhancement of the polypeptide activity of the present disclosure may be achieved by:
1) an increase in the intracellular copy number of a polynucleotide encoding a polypeptide;
2) replacement of the gene expression control region on the chromosome encoding a polypeptide with a sequence having strong activity;
3) modification of the nucleotide sequence encoding the start codon or 5′-UTR region of a gene transcript encoding a polypeptide;
4) modification of the amino acid sequence of a polypeptide to enhance the polypeptide activity;
5) modification of a polynucleotide sequence encoding a polypeptide to enhance the polypeptide activity (for example, modification of the polynucleotide sequence of a polypeptide gene to encode a polypeptide that has been modified to enhance the activity of the polypeptide);
6) introduction of an exogenous polypeptide exhibiting the activity of a polypeptide or an exogenous polynucleotide encoding the same;
7) codon optimization of a polynucleotide encoding a polypeptide;
8) modification or chemical modification of an exposed site selected through analysis of the tertiary structure of a polypeptide: or
9) a combination of two or more selected from 1) to 8) above, but is not particularly limited thereto.

In additional embodiments,
1) the increase in the intracellular copy number of a polynucleotide encoding a polypeptide may be achieved by introduction into a host cell of a vector capable of replicating and functioning independently of a host, to which a polynucleotide encoding the polypeptide is operably linked. Alternatively, the increase may be achieved by introduction of one or two or more copies of a polynucleotide encoding the polypeptide into a chromosome in a host cell. The introduction into a chromosome may be performed by introducing a vector capable of inserting the polynucleotide into the chromosome in a host cell into the host cell, but is not limited thereto.
2) The replacement of the gene expression control region (or expression control sequence) on the chromosome encoding a polypeptide with a sequence having strong activity may be, for example, occurrence of mutation in the sequence by deletion, insertion, non-conservative or conservative substitution or a combination thereof to further enhance the activity of the expression control region: or replacement with a sequence having stronger activity. The expression control region may include, but is not particularly limited to, a promoter, an operator sequence, a sequence encoding a ribosome binding site, and a sequence for regulating the termination of transcription and translation. As an example, the replacement may be to replace the original promoter with a strong promoter, but is not limited thereto.

Examples of known strong promoters include, but are not limited to, CJ1 to CJ7 promoters (U.S. Pat. No. 7,662,943 B2), lac promoter, trp promoter, trc promoter, tac promoter, lambda phage PR promoter, PL promoter, tet promoter, gapA promoter, SPL7 promoter, SPL13 (sm3) promoter (U.S. Pat. No. 10,584,338 B2), 02 promoter (U.S. Pat. No. 10,273,491 B2), tkt promoter, and yccA promoter.
3) The modification of the nucleotide sequence encoding the start codon or 5′-UTR region of a gene transcript encoding a polypeptide may be, for example, but is not limited to, substitution with a nucleotide sequence encoding another start codon having a higher polypeptide expression rate compared to the endogenous start codon.
4) and 5) The modification of the amino acid sequence or polynucleotide sequence may be, but is not limited to, occurrence of mutation in the amino acid sequence of a polypeptide or the polynucleotide sequence encoding the polypeptide by deletion, insertion, non-conservative or conservative substitution, or a combination thereof so that the activity of the polypeptide is enhanced, or replacement of the sequence with an amino acid sequence or polynucleotide sequence modified to have stronger activity or an amino acid sequence or polynucleotide sequence modified to increase activity. The replacement may be specifically performed by inserting a polynucleotide into a chromosome by homologous recombination, but is not limited thereto. The vector used at this time may further include a selection marker for confirming chromosome insertion.
6) The introduction of an exogenous polynucleotide exhibiting the activity of a polypeptide may be introduction of an exogenous polynucleotide encoding a polypeptide exhibiting activity the same as/similar to that of the polypeptide into a host cell. The exogenous polynucleotide is not limited in origin or sequence as long as it exhibits activity the same as/similar to that of the polypeptide. As the method used for the introduction, a known transformation method may be appropriately selected by those skilled in the art. As the introduced polynucleotide is expressed in the host cell, a polypeptide may be produced and its activity may be increased.
7) The codon optimization of a polynucleotide encoding a polypeptide may be codon optimization of the endogenous polynucleotide so that the transcription or translation is increased in a host cell, or codon optimization of the exogenous polynucleotide so that the optimized transcription and translation are performed in a host cell.
8) The modification or chemical modification of an exposed site selected through analysis of the tertiary structure of a polypeptide may be, for example, determining a template protein candidate according to the degree of sequence similarity by comparing the sequence information of the polypeptide to be analyzed with a database in which sequence information of known proteins is stored, confirming the structure based on this, selecting an exposed site to be modified or chemically modified, selecting the exposed site to be modified or chemically modified, and modifying or chemically modifying the same.

Such enhancement of the polypeptide activity may be an increase in the activity or concentration expression level of the corresponding polypeptide compared to the activity or concentration of the polypeptide expressed in the wild-type microorganism or microorganism before modification, or an increase in the amount of product produced from the polypeptide, but is not limited thereto.

The modification of part or all of the polynucleotide in the microorganism according to some embodiments of the present disclosure may be induced by (a) homologous recombination using a vector for chromosome insertion into microorganisms or genome editing using engineered nuclease (e.g., CRISPR-Cas9) and/or (b) light such as ultraviolet light and radiation and/or chemical treatment, but is not limited thereto. The method for modifying part or all of the gene may include a method by DNA recombination technology. For example, by injecting a nucleotide sequence or vector including a nucleotide sequence homologous to the target gene into the microorganism to cause homologous recombination, part or all of the gene may be deleted. The injected nucleotide sequence or vector may include a dominant selection marker, but is not limited thereto.

Another aspect of the present disclosure is to provide a vector comprising the polynucleotide described above.

The vector according to some embodiments of the present disclosure may include a DNA construct containing a nucleotide sequence of a polynucleotide encoding a target polypeptide operably linked to a suitable expression control region (or expression control sequence) so that the target polypeptide can be expressed in a suitable host. The expression control region may include a promoter capable of initiating transcription, an optional operator sequence for regulating such transcription, a sequence encoding a suitable mRNA ribosome binding site, and a sequence regulating the termination of transcription and translation. After transformation into an appropriate host cell, the vector may replicate or function independently of the host genome, and may be integrated into the genome itself.

The vector used in the present disclosure is not particularly limited, and any vector known in the art may be used. Examples of commonly used vectors include natural or recombinant plasmids, cosmids, viruses and bacteriophages. For example, pWE15, M13, MBL3, MBL4, IXII, ASHII, APII, t10, t11, Charon4A, and Charon21A may be used as a phage vector or cosmid vector, and a pDZ system, a pBR system, a pUC system, a pBluescript II system, a pGEM system, a pTZ system, a pCL system, and a pET system may be used as a plasmid vector. Specifically, pDZ, pDC, pDCM2, pACYC177, pACYC184, pCL, pECCG117, pUC19, pBR322, pMW118, pCC1BAC vectors and the like may be used.

As an example, a polynucleotide encoding a target polypeptide may be inserted into a chromosome through a vector for intracellular chromosome insertion. The insertion of a polynucleotide into a chromosome may be performed by any method known in the art, for example, homologous recombination, but is not limited thereto. A selection marker for confirming chromosome insertion may be further included. The selection marker is used to select cells transformed with a vector, that is, to confirm the insertion of a target nucleic acid molecule, and markers that confer selectable phenotypes such as drug resistance, auxotrophy, resistance to cytotoxic agents, and expression of surface polypeptides may be used. In an environment treated with a selective agent, only the cells expressing the selection marker can survive or exhibit other expression traits, and thus the transformed cells can be selected.

As used herein, the term “transformation” refers to introducing a vector including a polynucleotide encoding a target polypeptide into a host cell or microorganism so that the polypeptide encoded by the polynucleotide can be expressed in the host cell. The transformed polynucleotide may include both of a transformed polynucleotide that is located by being inserted into the chromosome of the host cell and a transformed polynucleotide that is located outside the chromosome as long as they can be expressed in the host cell. In addition, the polynucleotide includes DNA and/or RNA encoding a target polypeptide. The polynucleotide may be introduced in any form as long as it can be introduced into and expressed in a host cell. For example, the polynucleotide may be introduced into a host cell in the form of an expression cassette, which is a gene construct including all elements required for self-expression. The expression cassette may usually include a promoter operably linked to the polynucleotide, a transcription termination signal, a ribosome binding site, and a translation termination signal. The expression cassette may be in the form of an expression vector capable of self-replication. In addition, the polynucleotide may be introduced into a host cell in its own form and operably linked to a sequence required for expression in the host cell, but is not limited thereto.

In addition, the term “operably linked” as used herein means that a promoter sequence, which initiates and mediates transcription of the polynucleotide encoding the target polypeptide of the present application, and the polynucleotide sequence are functionally linked to each other.

The microorganism according to some embodiments of the present disclosure may be a microorganism in which the activity of the protein described herein or a polynucleotide encoding the same is weakened: or a microorganism (for example, a recombinant microorganism) genetically modified through a vector so that the activity of the protein described herein or a polynucleotide encoding the same is weakened, but is not limited thereto. The vector is as described above.

The microorganism according to some embodiments of the present disclosure may be a microorganism having L-glutamic acid producing ability.

The microorganism according to some embodiments of the present disclosure may be a microorganism naturally having the L-glutamic acid producing ability or a microorganism to which L-glutamic acid producing ability is imparted because the activity of the protein described herein or a polynucleotide encoding the same is weakened in the parent strain that does not have L-glutamic acid producing ability, but is not limited thereto.

As an example, the recombinant microorganism of the present disclosure is a microorganism, which is transformed through a vector so that the activity of the protein described herein or a polynucleotide encoding the same is weakened and thus exhibits weakened activity of the protein described herein or a polynucleotide encoding the same, and may include all microorganisms that can produce L-glutamic acid because the activity of the protein described herein or a polynucleotide encoding the same is weakened therein.

For the purpose according to some embodiments of the present disclosure, the recombinant microorganism according to some embodiments of the present disclosure may be a microorganism having increased L-glutamic acid producing ability compared to a natural wild-type microorganism or a microorganism, which contains the protein described herein or a polynucleotide encoding the same and produces L-glutamic acid because the activity of the protein described herein or a polynucleotide encoding the same is weakened in the natural wild-type microorganism or the microorganism, which contains the protein described herein or a polynucleotide encoding the same and produces L-glutamic acid, but is not limited thereto. For example, the unmodified microorganism in which the activity of the protein described herein is not weakened as the target strain for comparing the increase in the L-glutamic acid producing ability may be a Corynebacterium glutamicum ATCC13869 strain, in which the odhA gene is deleted and is known as an L-glutamic acid producing strain, or a Corynebacterium glutamicum BL2 strain (KFCC11074, Korean Patent No. 10-0292299) known as an L-glutamic acid producing NTG-mutant strain, but is not limited thereto.

As an example, the L-glutamic acid producing ability of the recombinant strain having increased producing ability may be increased by about 0.3% or more, specifically by about 0.5% or more, about 1% or more, about 2% or more, about 3% or more, about 4% or more, about 5% or more, about 6% or more, about 7% or more, about 8% or more, about 9% or more, about 10% or more, about 10.7% or more, about 11% or more, about 12% or more, about 12.3% or more, about 13% or more, about 14% or more, about 14.4% or more, about 15% or more, about 15.1% or more, about 16% or more, or about 16.9% or more (the upper limit thereof is not particularly limited and the producing ability may be increased by, for example, about 200% or less, about 150% or less, about 100% or less, about 50% or less, about 40% or less, about 30% or less, or about 20% or less) compared to the L-glutamic acid producing ability of the parent strain before mutation or unmodified microorganism, but the producing ability is not limited thereto as long as it has an increased amount of + value compared to the producing ability of the parent strain before mutation or an unmodified microorganism thereof. In another example, the L-glutamic acid producing ability of the microorganism having the increased producing ability may be increased by about 1.005 times or more, about 1.01 times or more, about 1.02 times or more, about 1.03 times or more, about 1.04 times or more, about 1.05 times or more, about 1.06 times or more, about 1.07 times or more, about 1.08 times or more, about 1.09 times or more, about 1.10 times or more, about 1.107 times or more, about 1.11 times or more, about 1.12 times or more, about 1.123 times or more, about 1.13 times or more, about 1.14 times or more, about 1.144 times or more, about 1.15 times or more, about 1.151 times or more, about 1.16 times or more, or about 1.169 times or more (the upper limit thereof is not particularly limited and the producing ability may be increased by, for example, about 10 times or less, about 5 times or less, about 3 times or less, or about 2 times or less) compared to the L-glutamic acid producing ability of the parent strain before mutation or an unmodified microorganism thereof, but is not limited thereto.

As used herein, the term “unmodified microorganism” does not exclude strains containing mutations that may occur naturally in microorganisms, and may refer to a wild-type strain or a natural strain itself, or a strain before the trait is changed by genetic mutation due to natural or artificial factors. For example, the unmodified microorganism may refer to a strain in which the activity of the protein described herein described herein is not weakened or has not yet been weakened. The “unmodified microorganism” may be used interchangeably with “strain before modification”, “microorganism before modification”, “unmutated strain”, “unmodified strain”, “unmutated microorganism”, or “reference microorganism”.

As another example of the present disclosure, the microorganism of the present disclosure may be Corynebacterium glutamicum, Corynebacterium stationis, Corynebacterium crudilactis, Corynebacterium Corynebacterium efficiens, Corynebacterium callunae, Corynebacterium singulare, Corynebacterium halotolerans, Corynebacterium striatum, Corynebacterium ammoniagenes, Corynebacterium pollutisoli, Corynebacterium imitans, Corynebacterium testudinoris or Corynebacterium flavescens, and specifically Corynebacterium glutamicum, but is not limited thereto.

As another example, the recombinant microorganism of the present disclosure may be a microorganism of which the L-glutamic acid producing ability is enhanced as the activity of part of the protein in the L-glutamic acid biosynthetic pathway is additionally enhanced or the activity of part of the protein in the L-glutamic acid decomposition pathway is additionally inactivated.

Specifically, the microorganism according to some embodiments of the present disclosure may be a microorganism in which the OdhA protein is additionally inactivated or the odhA gene is additionally deleted. More specifically, the microorganism according to some embodiments of the present disclosure may be Corynebacterium glutamicum in which the OdhA protein is inactivated in Corynebacterium glutamicum ATCC13869 or a microorganism in which the odhA gene is deleted in the Corynebacterium glutamicum ATCC13869. The sequence of the “OdhA protein” can be obtained from NCBI GenBank, a known database, and the OdhA protein may include, for example, the amino acid sequence of NCBI Sequence ID WP_060564343.1; additionally, the odhA gene may include the nucleotide sequence of NCBI GenBank BBD29_06050, and may include those exhibiting the same activity without being limited thereto.

However, the OdhA protein inactivation or odhA gene deletion is an example, and the microorganism is not limited thereto, and the microorganism described herein may be a microorganism in which the protein activity in various known L-glutamic acid biosynthetic pathways is enhanced or the protein activity in the decomposition pathway is inactivated or weakened.

Another aspect of the present disclosure provides a method for producing L-glutamic acid, comprising culturing the microorganism of the present disclosure in a medium.

The method for producing L-glutamic acid according to some embodiments of the present disclosure may comprises culturing a microorganism in which the activity of the protein described herein or a polynucleotide encoding the same is weakened: or a microorganism of the genus Corynebacterium genetically modified through a vector so that the activity of the protein described herein or a polynucleotide encoding the same is weakened in a medium.

In the present disclosure, the term “culture” means growing the microorganism described herein under properly controlled environmental conditions. The culture process according to some embodiments of the present disclosure may be performed under appropriate medium and culture conditions known in the art. Such a culture process may be easily adjusted and used by those skilled in the art depending on the selected microorganism. Specifically, the culture may be batch, continuous and/or fed-batch culture, but is not limited thereto.

As used herein, the term “medium” refers to a material in which nutrients required for culturing the microorganism described herein are mixed as a main component, and supplies nutrients and growth factors, including water, which are essential for survival and growth. Specifically, as the medium and other culture conditions used for culturing the microorganism described herein, any medium may be used without particular limitation as long as it is a medium used for culturing conventional microorganisms, but the microorganism described herein may be cultured in a conventional medium containing appropriate carbon sources, nitrogen sources, phosphorus sources, inorganic compounds, amino acids, vitamins and/or the like under an aerobic condition while the temperature, pH, and the like are adjusted.

Specifically, a culture medium for microorganisms of the genus Corynebacterium may be found in the literature [“Manual of Methods for General Bacteriology” by the American Society for Bacteriology (Washington D.C., USA, 1981)].

In the present disclosure, the carbon sources include carbohydrates such as glucose, saccharose, lactose, fructose, sucrose, and maltose: sugar alcohols such as mannitol and sorbitol: organic acids such as pyruvic acid, lactic acid, and citric acid; and amino acids such as glutamic acid, methionine, and lysine. In addition, natural organic nutrients such as starch hydrolysates, molasses, blackstrap molasses, rice bran, cassava, sugar cane waste and corn steep liquor may be used, specifically, carbohydrates such as glucose and sterilized pre-treated molasses (namely, molasses converted to reducing sugar) may be used, and appropriate amounts of other carbon sources may be variously used without limitation. These carbon sources may be used singly or in combination of two or more, but the manner of use is not limited thereto.

As the nitrogen sources, inorganic nitrogen sources such as ammonia, ammonium sulfate, ammonium chloride, ammonium acetate, ammonium phosphate, ammonium carbonate, and ammonium nitrate; and organic nitrogen sources such as amino acids such as glutamic acid, methionine, and glutamine, peptone, NZ-amine, Beef extract, yeast extract, malt extract, corn steep liquor, casein hydrolysates, fish or decomposition products thereof, and defatted soy bean cake or decomposition products thereof may be used. These nitrogen sources may be used singly or in combination of two or more, but the manner of use is not limited thereto.

The phosphorus sources may include monobasic potassium phosphate and dibasic potassium phosphate or monobasic sodium phosphate and dibasic sodium phosphate. As the inorganic compounds, sodium chloride, calcium chloride, iron chloride, magnesium sulfate, iron sulfate, manganese sulfate, calcium carbonate, and the like may be used. In addition to these, amino acids, vitamins, suitable precursors and/or the like may be contained. These components or precursors may be added to the medium batchwise or continuously. However, the manner of addition is not limited thereto.

During the culture of the microorganism according to some embodiments of the present disclosure, compounds such as ammonium hydroxide, potassium hydroxide, ammonia, phosphoric acid, sulfuric acid, and the like may be added to the medium in an appropriate manner to adjust the pH of the medium. During the culture, an antifoaming agent such as fatty acid polyglycol ester may be used to suppress bubble formation. Oxygen or oxygen-containing gas may be injected into the medium in order to maintain the aerobic state of the medium: or gas may not be injected or nitrogen, hydrogen or carbon dioxide gas may be injected in order to maintain the anaerobic and microaerobic conditions, but the control of atmosphere is not limited thereto.

In the culture according to some embodiments of the present disclosure, the culture temperature may be maintained at 20° C. to 45° C., specifically, at 25° C. to 40° C., and the culture may be conducted for about 10 to 160 hours, but the culture conditions are not limited thereto.

L-Glutamic acid produced by the culture according to some embodiments of the present disclosure may be secreted into the medium or may remain in the cells.

The method for producing L-glutamic acid according to some embodiments of the present disclosure may further comprise a step of preparing the microorganism described herein, a step of preparing a medium for culturing the microorganism, or a combination thereof (in any order), for example, before the culture step.

The method for producing L-glutamic acid according to some embodiments of the present disclosure may further comprise a step of recovering L-glutamic acid from the medium after culture (medium subjected to culture) or the cultured microorganism. The recovery step may be further comprised after the culture step.

The recovery may be collection of desired L-glutamic acid using a suitable method known in the art according to the method for culturing a microorganism described herein, for example, a batch, continuous or fed-batch culture method. For example, centrifugation, filtration, treatment with a crystallized protein precipitating agent (salting-out method), extraction, sonication, ultrafiltration, dialysis, various kinds of chromatography such as molecular sieve chromatography (gel filtration), adsorption chromatography, ion-exchange chromatography, and affinity chromatography, HPLC, or any combination thereof may be used, and the desired L-glutamic acid may be recovered from the medium or microorganism using a suitable method known in the art.

The method for producing L-glutamic acid according to some embodiments of the present disclosure may further comprise a purification step. The purification may be performed using a suitable method known in the art. In an example, when the method for producing L-glutamic acid of the present disclosure comprises both a recovery step and a purification step, the recovery step and the purification step may be performed continuously or discontinuously in any order, or may be performed simultaneously or by being integrated into one step, but the manner of performance is not limited thereto.

In the method according to some embodiments of the present disclosure, the protein, polynucleotide, vector, microorganism and the like are as described in the other aspects above.

Still another aspect of the present disclosure provides a composition for L-glutamic acid production, comprising a microorganism of the genus Corynebacterium in which the activity of the protein described herein is weakened: a medium in which the microorganism has been cultured; or a combination thereof.

The composition according to some embodiments of the present disclosure may further contain arbitrary suitable excipients commonly used in compositions for L-glutamic acid production, and such excipients may include, for example, but are not limited to, preservatives, wetting agents, dispersing agents, suspending agents, buffering agents, stabilizing agents, and isotonic agents.

Still another aspect of the present disclosure provides a method for producing a microorganism of the genus Corynebacterium, comprising weakening the activity of the protein of the present disclosure.

Still another aspect of the present disclosure provides use for producing L-glutamic acid of a microorganism of the genus Corynebacterium in which the activity of the protein of the present disclosure is weakened.

The protein, weakening, microorganism of the genus Corynebacterium, and the like are as described in the other aspects.

### Advantageous Effects

The L-glutamic acid producing microorganism of the genus Corynebacterium in which the activity of a protein is weakened of the present disclosure can produce L-glutamic acid at a high yield, and can be thus usefully utilized for industrial production of L-glutamic acid.

### EXAMPLES

Hereinafter, the present application will be described in more detail with reference to Examples. However, the following Examples are merely preferred embodiments for illustrating the present application, and therefore, the scope of the present application is not intended to be limited thereto. Meanwhile, technical matters not described in this specification can be sufficiently understood and easily implemented by those skilled in the art of the present application or similar technical fields.

### Example 1. Preparation of Recombinant Vector for Changing Start Codon of BBD29_13140 Gene

The change in L-glutamic acid producing ability was confirmed when the start codon of the BBD29_13140 gene (SEQ ID NO: 2) on the chromosome in a strain of the genus Corynebacterium was changed from ATG to GTG and TTG, respectively.

Specifically, in order to prepare a vector for changing a start codon, gene fragments were obtained by performing PCR using the chromosomal DNA of Corynebacterium glutamicum ATCC13869 as a template along with a primer pair of SEQ ID NOS: 3 and 4 and a primer pair of SEQ ID NOS: 5 and 6. Solg™ Pfu-X DNA polymerase was used as a polymerase, and the PCR amplification was performed as follows: denaturation at 95° C. for 5 minutes: 30 cycles of denaturation at 95° C. for 30 seconds, annealing at 55° C. for 30 seconds, and extension at 72° C. for 60 seconds; and extension at 72° C. for 5 minutes.

The amplified gene fragments were cloned into the pDCM2 (KR Patent Application Publication No. 10-2020-0136813), which was a chromosomal transformation vector digested with SmaI restriction enzyme, using the Gibson assembly method (DG Gibson et al., Nature Methods, Vol. 6 No. 5, May 2009, NEBuilder HiFi DNA Assembly Master Mix) to obtain a recombinant plasmid, and the resulting plasmid was named pDCM2-BBD29_13140(a1g) vector. The cloning was performed by mixing the Gibson assembly reagent and each gene fragment in the calculated numbers of moles and then storing the mixture at 50° C. for one hour.

Gene fragments were obtained by performing PCR using the chromosomal DNA of Corynebacterium glutamicum ATCC13869 as a template along with a primer pair of SEQ ID NOS: 3 and 7 and a primer pair of SEQ ID NOS: 8 and 6. Solg™ Pfu-X DNA polymerase was used as a polymerase, and the PCR amplification was performed as follows: denaturation at 95° C. for 5 minutes: 30 cycles of denaturation at 95° C. for 30 seconds, annealing at 55° C. for 30 seconds, and extension at 72° C. for 60 seconds; and extension at 72° C. for 5 minutes.

The amplified gene fragments were cloned into the pDCM2, which was a chromosomal transformation vector digested with SmaI restriction enzyme, using the Gibson assembly method to obtain a recombinant plasmid, and the resulting plasmid was named pDCM2-BBD29_13140(a1t) vector. The cloning was performed by mixing the Gibson assembly reagent and each gene fragment in the calculated numbers of moles and then storing the mixture at 50° C. for one hour.

The thus-prepared pDCM2-BBD29_13140(a1g) and pDCM2-BBD29_13140(a1t) vectors were introduced into strains in the following Examples.

### Example 2. Preparation of Corynebacterium glutamicum Strain in which Start Codon of BBD29_13140 Gene is Changed

### Example 2-1. Preparation of Corynebacterium glutamicum Strain Having L-Glutamic Acid Producing Ability Derived from Wild-Type Corynebacterium glutamicum

To prepare a strain having L-glutamic acid producing ability derived from Corynebacterium glutamicum ATCC13869, a Corynebacterium glutamicum ATCC13869ΔodhA strain, in which odhA gene was deleted, was prepared based on the literature (Appl Environ Microbiol. 2007 February: 73 (4): 1308-19. Epub, Dec. 8, 2006). The sequence of OdhA protein (GenBank Accession No. WP_060564343.1) was obtained from GenBank of the NCBI, which is a known database.

Specifically, for deletion of odhA gene, an upstream region and a downstream region of odhA gene were obtained by performing PCR using the chromosomal DNA of Corynebacterium glutamicum ATCC13869 as a template along with a primer pair of SEQ ID NOS: 9 and 10 and a primer pair of SEQ ID NOS: 11 and 12, respectively. Solg™ Pfu-X DNA polymerase was used as a polymerase, and the PCR amplification was performed as follows: denaturation at 95° C. for 5 minutes: 30 cycles of denaturation at 95° C. for 30 seconds, annealing at 58° C. for 30 seconds, and extension at 72° C. for 60 seconds; and extension at 72° C. for 5 minutes.

The amplified upstream and downstream regions of odhA gene were cloned into the pDCM2, which is a chromosomal transformation vector digested with SmaI restriction enzyme, using the Gibson assembly method to obtain a recombinant plasmid, and the resulting plasmid was named pDCM2-ΔodhA. The cloning was performed by mixing the Gibson assembly reagent and each gene fragment in the calculated numbers of moles and then storing the mixture at 50° C. for one hour.

The thus-prepared pDCM2-ΔodhA vector was transformed into the Corynebacterium glutamicum ATCC13869 strain by electroporation and then subjected to secondary crossover to obtain a strain in which the odhA gene was deleted on the chromosome. The deletion of odhA gene was confirmed through genome sequencing and PCR using a primer pair of SEQ ID NO: 13 and SEQ ID NO: 14, and the prepared strain was named ATCC13869ΔodhA.

### Example 2-2. Preparation of Strains in which BBD29_13140 Gene is Weakened and which has L-Glutamic Acid Producing Ability Derived from Corynebacterium glutamicum

The pDCM2-BBD29_13140(a1g) vector and pDCM2-BBD29_13140(a1t) vector prepared in Example 1 were each introduced into the Corynebacterium glutamicum ATCC13869ΔodhA strain prepared in Example 2-1, and the effect of the introduction on L-glutamic acid producing ability was confirmed.

Each of the two vectors was transformed into the Corynebacterium glutamicum ATCC13869ΔodhA strain by electroporation and then subjected to secondary crossover to obtain strains, in which the start codon of the BBD29_13140 gene was changed from ATG to GTG and TTG, respectively. The gene manipulation was confirmed through genome sequencing and PCR using a primer pair of SEQ ID NO: 15 and SEQ ID NO: 16, and the prepared strains were each named ATCC13869ΔodhA-BBD29_13140(a1g) and ATCC13869ΔodhA-BBD29_13140(a1t).

The thus-prepared ATCC13869ΔodhA-BBD29_13140(a1g) and ATCC13869ΔodhA-BBD29_13140(a1t) strains were cultured by the following method using the ATCC13869ΔodhA strain as the control to confirm their L-glutamic acid producing abilities.

The strains were inoculated on a plate medium consisting of the seed medium below and cultured at 30° C. for 20 hours. These strains were then inoculated into a 250 mL corner-baffled flask containing 25 mL of the production medium below using one inoculation loop, and cultured at 30° C. for 40 hours with shaking at 200 rpm.

### <Seed Medium>

Glucose 1%, Beef extract 0.5%, Polypeptone 1%, Sodium chloride 0.25%, Yeast extract 0.5%, Agar 2%, Urea 0.2%, pH 7.2

### <Production Medium>

Raw sugar 6%, Calcium carbonate 5%, Ammonium sulfate 2.25%, Monopotassium phosphate 0.1%, Magnesium sulfate 0.04%, Iron sulfate 10 mg/L, Thiamine-HCl 0.2 mg/L, Biotin 50 μg/L

After completion of the culture, L-glutamic acid producing ability was measured by high-performance liquid chromatography (HPLC), and the measurement results are shown in Table 1 below.

TABLE 1

| | | Rate of Increase |
|---|---|---|
| | L-Glutamic Acid | in L-Glutamic Acid |
| Name of Strains | Concentration (g/L) | Concentration (%) |
| ATCC13869ΔodhA | 1.99 | — |
| ATCC13869ΔodhA- | 2.13 | 107.0% |
| BBD29_13140(a1g) | | |
| ATCC13869ΔodhA- | 2.29 | 115.1% |
| BBD29_13140(a1t) | | |

As shown in Table 1 above, it was confirmed that the concentration of L-glutamic acid was increased by about 7% and 15.1% in ATCC13869ΔodhA-BBD29_13140(a1g) and ATCC13869ΔodhA-BBD29_13140(a1t) strains, respectively, in which the start codon of the BBD29_13140 gene was weakened from ATG to GTG or TTG, compared to that in the ATCC13869ΔodhA strain derived from a wild-type.

### Example 3. Preparation of Recombinant Vector in which Ribosome Binding Site (RBS) of BBD29_13140 Gene is Changed

In order to weaken the BBD29_13140 gene on the chromosome in a strain of the genus Corynebacterium, the Shine-Dalgarno (SD) sequence was predicted from the nucleotide sequences containing the upstream 35 base pairs of the gene and 35 base pairs from the N-terminus of the open reading frame (ORF).

Based on the nucleotide sequences, a total of four ribosome binding site candidate groups (i.e., RBS1, RBS2, RBS3, and RBS4) were predicted through the RBS calculator (github). As a result, it was confirmed that the four ribosome binding site candidate groups are sequences whose expression is predicted to be reduced by 20%, 60%, 80%, and 90%, respectively, compared to the existing ribosome binding site.

The predicted ribosome binding sites are as shown in Table 2 below.

TABLE 2

| | | | Predicted |
|---|---|---|---|
| SEQ | | Shine-Dalgarno | Expression |
| ID NO | Name | Sequence | Level (%) |
| 34 | original SD | CTAGATTGG | 100% |
| 35 | RBS1 | CAAGGCCGG | 80% |
| 36 | RBS2 | CGTGACAGG | 40% |
| 37 | RBS3 | CTTCGCTGG | 20% |
| 38 | RBS4 | GTCGATTTC | 10% |

In order to confirm whether the L-glutamic acid producing ability is enhanced when the gene is weakened by changing the ribosome binding site of BBD29_13140 on the chromosome in a strain of the genus Corynebacterium, recombinant vectors, in which the ribosome binding sites for each of the four previously predicted ribosome binding sites was changed, were prepared.

Specifically, gene fragments were obtained by performing PCR using the chromosomal DNA of wild-type Corynebacterium glutamicum ATCC13869 as a template along with primer pairs of SEQ ID NO: 17 and SEQ ID NO: 18; SEQ ID NO: 19 and SEQ ID NO: 20; SEQ ID NO: 17 and SEQ ID NO: 21; SEQ ID NO: 22 and SEQ ID NO: 20; SEQ ID NO: 17 and SEQ ID NO: 23: SEQ ID NO: 24 and SEQ ID NO: 20; SEQ ID NO: 17 and SEQ ID NO: 25; and SEQ ID NO: 26 and SEQ ID NO: 20. Solg™ Pfu-X DNA polymerase was used as a polymerase, and the PCR amplification was performed as follows: denaturation at 95° C. for 5 minutes: 30 cycles of denaturation at 95° C. for 30 seconds, annealing at 55° C. for 30 seconds, and extension at 72° C. for 60 seconds; and extension at 72° C. for 5 minutes.

The amplified gene fragments were cloned into the pDCM2, which is a chromosomal transformation vector digested with SmaI restriction enzyme, using the Gibson assembly method to obtain recombinant vectors, and the resulting vectors were each named pDCM2-RBS1 vector, pDCM2-RBS2 vector, pDCM2-RBS3 vector, and pDCM2-RBS4 vector, respectively. The cloning was performed by mixing the Gibson assembly reagent and each of the gene fragments in the calculated numbers of moles and then storing the mixture at 50° C. for one hour.

### Example 4. Preparation of Recombinant Strain for Changing Ribosome Binding Site (RBS) of BBD29_13140 Gene

The pDCM2-RBS1, pDCM2-RBS2, pDCM2-RBS3, and pDCM2-RBS4 vectors prepared in Example 3 were introduced into the Corynebacterium glutamicum ATCC13869ΔodhA strain prepared in Example 2-1, and the effect of the introduction on L-glutamic acid producing ability was confirmed.

Specifically, each of the four vectors was transformed into the Corynebacterium glutamicum ATCC13869ΔodhA strain by electroporation and then subjected to secondary crossover to obtain strains, in which the ribosome binding sequence of the BBD29_13140 gene was changed to RBS1, RBS2, RBS3, and RBS4, respectively.

The gene manipulation was confirmed through genome sequencing and PCR using a primer pair of SEQ ID NO: 27 and SEQ ID NO: 28, which were able to amplify the homologous recombinant upstream and downstream regions, respectively, and the resulting strains were each named ATCC13869ΔodhA-RBS1, ATCC13869ΔodhA-RBS2, ATCC13869ΔodhA-RBS3, and ATCC13869ΔodhA-RBS4.

The thus-prepared ATCC13869ΔodhA-RBS1, ATCC13869ΔodhA-RBS2, ATCC13869ΔodhA-RBS3, and ATCC13869ΔodhA-RBS4 strains were cultured by the same method as in Example 2-2 using the ATCC13869ΔodhA strain as the control to confirm their L-glutamic acid producing abilities.

After completion of the culture, the L-glutamic acid producing ability was measured by high-performance liquid chromatography (HPLC), and the measurement results are shown in Table 3 below.

TABLE 3

| | | Rate of Increase |
|---|---|---|
| | L-Glutamic Acid | in L-Glutamic Acid |
| Name of Strains | Concentration (g/L) | Concentration (%) |
| ATCC13869ΔodhA | 1.87 | — |
| ATCC13869ΔodhA-RBS1 | 1.88 | 100.5% |
| ATCC13869ΔodhA-RBS2 | 2.07 | 110.7% |
| ATCC13869ΔodhA-RBS3 | 2.10 | 112.3% |
| ATCC13869ΔodhA-RBS4 | 2.14 | 114.4% |

As shown in Table 3 above, it was confirmed that the L-glutamic acid concentration was increased in the strains, in which the ribosome binding sequence of the BBD29_13140 gene was weakened, compared to that in the ATCC13869ΔodhA strain derived from a wild-type.

### Example 5. Preparation of Vector in which BBD29_13140 Gene is Deleted

In addition to the effect of weakening the BBD29_13140 gene on the chromosome in a strain of the genus Corynebacterium confirmed in Examples above, in order to confirm the effect of deletion of BBD29_13140 gene on L-glutamic acid producing ability, a deletion vector was prepared.

To prepare a recombinant vector capable of deleting the gene, gene fragments were obtained by performing PCR using the chromosomal DNA of Corynebacterium glutamicum ATCC13869 as a template along with a primer pair of SEQ ID NOS: 3 and 29 and a primer pair of SEQ ID NOS: 30 and 31. Solg™ Pfu-X DNA polymerase was used as a polymerase, and the PCR amplification was performed as follows: denaturation at 95° C. for 5 minutes; 30 cycles of denaturation at 95° C. for 30 seconds, annealing at 55° C. for 30 seconds, and extension at 72° C. for 60 seconds; and extension at 72° C. for 5 minutes.

The amplified gene fragments were each cloned into the pDCM2 vector, which was a chromosomal transformation vector digested with SmaI restriction enzyme, using the Gibson assembly method to obtain a recombinant plasmid, and the resulting plasmid was named pDCM2-ΔBBD29_13140. The cloning was performed by mixing the Gibson assembly reagent and each gene fragment in the calculated numbers of moles and then storing the mixture at 50° C. for one hour.

The thus-prepared pDCM2-ΔBBD29_13140 vector was introduced into strains in Examples below.

### Example 6. Preparation of Corynebacterium glutamicum Strain in which BBD29_13140 Gene is Deleted

The pDCM2-ΔBBD29_13140 vector prepared in Example 5 was introduced into the ATCC13869ΔodhA strains prepared in Example 2-1, and the effect of the introduction on L-glutamic acid producing ability was confirmed.

Specifically, the pDCM2-ΔBBD29_13140 vector was transformed into the Corynebacterium glutamicum ATCC13869ΔodhA strain by electroporation and then subjected to secondary crossover to obtain a strain in which the BBD29_13140 gene was deleted on the chromosome. The genetic manipulation was confirmed through genome sequencing and PCR using a primer pair of SEQ ID NO: 32 and SEQ ID NO: 33, and the resulting strain was named ATCC13869ΔodhAΔBBD29_13140.

The thus-prepared ATCC13869ΔodhAΔBBD29_13140 strain was cultured by the same method as in Example 2-2 using the ATCC13869ΔodhA strain as the control to confirm the L-glutamic acid producing ability.

After completion of the culture, the L-glutamic acid producing ability was measured by high-performance liquid chromatography (HPLC), and the measurement results are shown in Table 4 below.

TABLE 4

| | | Rate of Increase |
|---|---|---|
| | L-Glutamic Acid | in L-Glutamic Acid |
| Name of Strains | Concentration (g/L) | Concentration (%) |
| ATCC13869ΔodhA | 2.00 | 100.0% |
| ATCC13869ΔodhAΔBBD29_13140 | 0.99 | 49.5% |

As shown in Table 4 above, it was confirmed that the growth of the ATCC13869ΔodhAΔBBD29_13140 strain, in which BBD29_13140 gene was deleted, was inhibited and the L-glutamic acid concentration in the ATCC13869ΔodhAΔBBD29_13140 strain was decreased to a level of about 49.5% compared to that in the ATCC13869ΔodhA strain derived from a wild-type.

### Example 7. Preparation of Corynebacterium glutamicum Strain Having L-Glutamic Acid Producing Ability Derived from N-Methyl-N′-Nitro-N-Nitrosoguanidine (NTG)-Mutant Corynebacterium glutamicum Strain in which BBD29_13140 Gene is Weakened

In order to confirm whether the gene exhibits the same effect in a NTG-mutant strain derived from the genus Corynebacterium with increased L-glutamic acid producing ability, in addition to the wild-type strains derived from the genus Corynebacterium, the pDCM2-BBD29_13140(a1t) prepared in Example 1 was introduced into the KFCC11074 strain (Korean Patent No. 10-0292299), which was known as an L-glutamic acid-producing NTG-mutant strain, and the effect of the introduction on L-glutamic acid producing ability was confirmed.

The vector was transformed into the KFCC11074 strain by electroporation and then subjected to secondary crossover to obtain a strain in which the start codon of the BBD29_13140 gene was changed from ATG to TTG on the chromosome. The genetic manipulation was confirmed through genome sequencing and PCR using a primer pair of SEQ ID NO: 15 and SEQ ID NO: 16, and the obtained strain was named KFCC11074-BBD29_13140(a1t).

The thus-prepared KFCC11074-BBD29_13140(a1t) strain and Corynebacterium glutamicum KFCC11074 strain were subjected to a fermentation titer test by the method specified below.

The strains were inoculated on a plate medium consisting of the seed medium below and cultured at 30° C. for 20 hours. These strains were then inoculated into a 250 mL corner-baffled flask containing 25 mL of the production medium below using one inoculation loop, and cultured at 30° C. for 40 hours with shaking at 200 rpm.

### <Seed Medium>

Glucose 1%, Beef extract 0.5%, Polypeptone 1%, Sodium chloride 0.25%, Yeast extract 0.5%, Agar 2%, Urea 0.2%, pH 7.2

### <Production Medium>

Raw sugar 6%, Calcium carbonate 5%, Ammonium sulfate 2.25%, Monopotassium phosphate 0.1%, Magnesium sulfate 0.04%, Iron sulfate 10 mg/L, Thiamine-HCL 0.2 mg/L, Biotin 500 μg/L

After completion of the culture, the L-glutamic acid producing ability was measured by high-performance liquid chromatography (HPLC), and the measurement results are shown in Table 5 below.

TABLE 5

| | | Rate of Increase |
|---|---|---|
| | L-Glutamic Acid | in L-Glutamic Acid |
| Name of Strains | Concentration (g/L) | Concentration (%) |
| KFCC11074 | 6.70 | 100.0% |
| KFCC11074- | 7.83 | 116.9% |
| BBD29_13140(a1t) | | |

As shown in Table 5 above, it was confirmed that the L-glutamic acid concentration in the KFCC11074-BBD29_13140(a1t) strain, in which the start codon of the BBD29_13140 gene was weakened from ATG to TTG, was increased by about 16.9% compared to that in the KFCC11074 strain.

Based on the above description, those skilled in the art to which the present application pertains will understand that the present application may be implemented in other specific forms without changing the technical spirit or essential characteristics thereof. In this regard, it should be understood that the embodiments described above are illustrative in all respects and not restrictive. With regard to the scope of the present application, it should be construed that all changes and modifications derived from the meaning and scope of the claims described below and their equivalents rather than the above detailed description are included in the scope of the present application.

### Certain Embodiments

Embodiment 1-1. A microorganism of the genus Corynebacterium, wherein the activity of a protein including an amino acid sequence of SEQ ID NO: 1 is weakened compared to intrinsic activity.
Embodiment 1-2. The microorganism according to Embodiment 1-1, wherein the microorganism of the genus Corynebacterium has L-glutamic acid producing ability.
Embodiment 1-3. The microorganism according to Embodiment 1-1, wherein the protein is encoded by a polynucleotide set forth in a nucleotide sequence of SEQ ID NO: 2.
Embodiment 1-4. The microorganism according to Embodiment 1-1, wherein the microorganism of the genus Corynebacterium is Corynebacterium glutamicum.
Embodiment 1-5. The microorganism according to Embodiment 1-1, wherein the microorganism has increased L-glutamic acid producing ability compared to a parent strain or wild-type strain in which the activity of a protein including an amino acid sequence of SEQ ID NO: 1 is not weakened compared to intrinsic activity.
Embodiment 1-6. A method for producing L-glutamic acid, comprising culturing a microorganism of the genus Corynebacterium, in which activity of a protein including an amino acid sequence of SEQ ID NO: 1 is weakened compared to intrinsic activity, in a medium.
Embodiment 1-7. The production method according to Embodiment 1-6, wherein the protein is encoded by a polynucleotide set forth in a nucleotide sequence of SEQ ID NO: 2.
Embodiment 1-8. The production method according to Embodiment 1-6, wherein the weakening of activity of a protein is weakening of a polynucleotide set forth in a nucleotide sequence of SEQ ID NO: 2.
Embodiment 1-9. The production method according to Embodiment 1-6, wherein the microorganism of the genus Corynebacterium is Corynebacterium glutamicum.
Embodiment 1-10. The production method according to Embodiment 1-6, wherein the production method further comprises recovering L-glutamic acid from the cultured microorganism or medium.
Embodiment 2-1. A microorganism, in which an activity of a protein comprising a sequence having at least 85% sequence identity to the amino acid sequence of SEQ ID NO: 1 is weakened compared to an intrinsic activity of the microorganism.
Embodiment 2-2. A microorganism expressing a recombinant protein comprising an amino acid sequence having at least 85% sequence identity to the amino acid sequence of SEQ ID NO: 1.
Embodiment 2-3. The microorganism according to Embodiment 2-1 or 2, wherein the protein comprises an amino acid sequence having at least 95% sequence identity to the amino acid sequence of SEQ ID NO: 1.
Embodiment 2-4. The microorganism according to any one of Embodiments 2-1 to 2-3, wherein the protein comprises the amino acid sequence of SEQ ID NO: 1.
Embodiment 2-5. The microorganism according to any one of Embodiments 2-1 to 2-3, wherein the protein does not comprise the amino acid sequence of SEQ ID NO: 1.
Embodiment 2-6. The microorganism according to any one of Embodiments 2-1 to 2-3, wherein the protein has a sequence selected from the group consisting of NCBI Accession No. WP_060565206.1, QWQ85246.1, WP_065367112.1, WP_038585884.1, WP_040072989.1, WP_011265991.1, WP_074492394.1, ARV66101.1, WP_003853812.1, WP_006285921.1, WP_003862956.1, WP_044027349.1, BAF55605.1, WP_179205783.1, WP_172769145.1, WP_211439424.1, WP_179111498.1, WP_059289862.1, WP_185969420.1, WP_173673681.1, and QDQ21801.1.
Embodiment 2-7. The microorganism according to any one of the preceding claims, wherein the microorganism has an L-glutamic acid production ability.
Embodiment 2-8. The microorganism according to any one of the preceding claims, wherein the protein is encoded by a nucleotide molecule comprising a sequence having at least 85% sequence identity to the nucleotide sequence of SEQ ID NO: 2 or its degenerate sequence.
Embodiment 2-9. The microorganism according to any one of the preceding claims, wherein the protein is encoded by a nucleotide molecule comprising a sequence having at least 95% sequence identity to the nucleotide sequence of SEQ ID NO: 2 or its degenerate sequence.
Embodiment 2-10. The microorganism according to any one of the preceding claims, wherein the protein is encoded by a nucleotide molecule comprising a sequence having at least 85% sequence identity to the nucleotide sequence of SEQ ID NO: 2.
Embodiment 2-11. The microorganism according to any one of the preceding claims, wherein the protein is encoded by a nucleotide molecule comprising a sequence having at least 95% sequence identity to the nucleotide sequence of SEQ ID NO: 2.
Embodiment 2-12. The microorganism according to any one of the preceding claims, wherein the protein is encoded by a nucleotide molecule comprising the nucleotide sequence of SEQ ID NO: 2.
Embodiment 2-13. The microorganism according to any one of the preceding claims, wherein the protein is encoded by a nucleotide molecule not comprising the nucleotide sequence of SEQ ID NO: 2.
Embodiment 2-14. The microorganism according to any one of the preceding claims, wherein the microorganism is Corynebacterium.
Embodiment 2-15. The microorganism according to any one of the preceding claims, wherein the microorganism is selected from the group consisting of Corynebacterium glutamicum, Corynebacterium stationis, Corynebacterium crudilactis, Corynebacterium deserti, Corynebacterium efficiens, Corynebacterium callunae, Corynebacterium singulare, Corynebacterium halotolerans, Corynebacterium striatum, Corynebacterium ammoniagenes, Corynebacterium pollutisoli, Corynebacterium imitans, Corynebacterium testudinoris, Corynebacterium flavescens. Corynebacterium crenatum and Corynebacterium suranareeae.
Embodiment 2-16. The microorganism according to any one of the preceding claims, wherein the microorganism is Corynebacterium glutamicum.
Embodiment 2-17. The microorganism according to any one of the preceding claims, wherein the microorganism has an increased ability to produce L-glutamic acid compared to a parent strain or wild-type in which the activity of the protein comprising the amino acid sequence of SEQ ID NO: 1 is not weakened compared to the intrinsic activity.
Embodiment 2-18. The microorganism according to any one of the preceding claims, in which at least a part of a nucleotide sequence coding the protein is deleted compared to an intrinsic nucleotide sequence of the microorganism.
Embodiment 2-19. The microorganism according to Embodiments 2-18, in which a nucleotide sequence coding the protein is deleted.
Embodiment 2-20. The microorganism according to any one of the preceding claims, in which an expression control sequence for a gene encoding the protein is not an intrinsic sequence of the microorganism.
Embodiment 2-21. The microorganism according to Embodiment 2-20, wherein the expression control sequence comprises at least one deletion, insertion or substitution in the intrinsic expression control sequence to reduce an expression of the gene encoding the protein.
Embodiment 2-22. The microorganism according to Embodiment 2-20 or 21, wherein the expression control sequence comprises at least one sequence selected from the group consisting of a promoter sequence, operator sequence, a sequence coding a ribosome binding site, a sequence coding termination of transcription, and a sequence coding termination of translation.
Embodiment 2-23. The microorganism according to any one of the preceding claims, wherein the amino acid sequence is not an intrinsic amino acid sequence of the microorganism.
Embodiment 2-24. The microorganism according to Embodiment 2-23, wherein the amino acid sequence comprises at least one deletion, insertion or substitution in the intrinsic amino acid sequence to weaken the activity of the protein.
Embodiment 2-25. The microorganism according to any one of the preceding claims, in which a nucleotide sequence coding the protein is not an intrinsic nucleotide sequence of the microorganism.
Embodiment 2-26. The microorganism according to Embodiment 2-25, wherein the nucleotide sequence comprises at least one deletion, insertion or substitution in the intrinsic nucleotide sequence to weaken the activity of the protein.
Embodiment 2-27. The microorganism according to any one of the preceding claims, in which at least one sequence selected from the group consisting of a nucleotide sequence for a start codon, a nucleotide sequence for stop codon, Shine-Dalgamno sequence, and 5′-UTR region of a gene encoding the protein is not an intrinsic sequence of the microorganism.
Embodiment 2-28. The microorganism according to Embodiment 2-27, wherein the nucleotide sequence for the start codon of the gene encoding the protein is mutated compared to the intrinsic sequence of the microorganism.
Embodiment 2-29. The microorganism according to Embodiment 2-27 or 28, wherein the nucleotide sequence for a stop codon of the gene encoding the protein is mutated compared to the intrinsic sequence of the microorganism.
Embodiment 2-30. The microorganism according to any one of Embodiments 2-27 to 2-29, wherein an intrinsic gene encoding the protein comprises a start codon of ATG, and the ATG is replaced with GTG.
Embodiment 2-31. The microorganism according to any one of Embodiments 2-27 to 2-30, wherein an intrinsic gene encoding the protein comprises a start codon of ATG, and the ATG is replaced with TTG.
Embodiment 2-32. The microorganism according to any one of Embodiments 2-27 to 2-30, wherein the nucleotide sequence for a Shine-Dalgarno sequence of the gene encoding the protein is mutated compared to the intrinsic sequence of the microorganism.
Embodiment 2-33. The microorganism according to any one of Embodiments 2-27 to 2-32, wherein an intrinsic gene encoding the protein comprises a Shine-Dalgarno sequence of CTAGATTGG, and the CTAGATTGG is replaced with CAAGGCCGG.
Embodiment 2-34. The microorganism according to any one of Embodiments 2-27 to 2-32, wherein an intrinsic gene encoding the protein comprises a Shine-Dalgarno sequence of CTAGATTGG, and the CTAGATTGG is replaced with CGTGACAGG.
Embodiment 2-35. The microorganism according to any one of Embodiments 2-27 to 2-32, wherein an intrinsic gene encoding the protein comprises a Shine-Dalgarno sequence of CTAGATTGG, and the CTAGATTGG is replaced with CTTCGCTGG.
Embodiment 2-36. The microorganism according to any one of Embodiments 2-27 to 2-32, wherein an intrinsic gene encoding the protein comprises a Shine-Dalgarno sequence of CTAGATTGG, and the CTAGATTGG is replaced with GTCGATTTC.
Embodiment 2-37. The microorganism according to any one of the preceding claims, wherein the microorganism comprises an antisense oligonucleotide that complementarily binds to the transcript of a gene encoding the protein.
Embodiment 2-38. The microorganism according to Embodiment 2-37, wherein the antisense oligonucleotide comprises an antisense RNA.
Embodiment 2-39. The microorganism according to any one of the preceding claims, wherein a sequence complementary to a Shine-Dalgarno sequence of a gene encoding the protein is inserted in front of the Shine-Dalgarno sequence to form a secondary structure.
Embodiment 2-40. The microorganism according to Embodiment 2-39, wherein the secondary structure does not bind to the ribosome to reduce or delay mRNA translation.
Embodiment 2-41. The microorganism according to any one of the preceding claims, in which a promoter for reverse transcription engineering (RTE) is inserted at 3′ end of open reading frame (ORF) of a gene encoding the protein.
Embodiment 2-42. The microorganism according to Embodiment 2-41, wherein an antisense nucleotide molecule complementary to at least a part of a gene encoding the protein is produced, thereby weakening the activity of a protein.
Embodiment 2-43. The microorganism according to any one of the preceding claims, in which odhA gene is deleted.
Embodiment 2-44. The microorganism according to any one of the preceding claims, wherein the microorganism is ATCC13869 in which odhA gene is deleted.
Embodiment 2-45. The microorganism according to Embodiment 2-43 or 44, wherein the odhA gene comprises a nucleotide sequence encoding NCBI Sequence ID WP 060564343.1.
Embodiment 2-46. The microorganism according to Embodiment 2-43 or 44, wherein the odhA gene comprises NCBI GenBank BBD29_06050.
Embodiment 2-47. The microorganism according to any one of the preceding claims, the microorganism comprising an N-methyl-N′-nitro-N-nitrosoguanidine (NTG)-mutant strain.
Embodiment 2-48. Use of the microorganism of any one of the preceding claims for producing L-glutamic acid.
Embodiment 2-49. A method of producing L-glutamic acid, comprising culturing the microorganism of any one of Embodiments 2-1 to 2-45.
Embodiment 2-50. The method according to Embodiment 2-49, wherein the protein is encoded by the nucleotide sequence of SEQ ID NO: 2, and an activity of the nucleotide sequence of SEQ ID NO: 2 is weakened compared to the intrinsic activity.
Embodiment 2-51. The method according to Embodiment 2-49 or 50, wherein the production method further comprises recovering L-glutamic acid from the cultured microorganism or medium.
Embodiment 2-52. The method according to any one of Embodiments 2-49 to 2-51, wherein the production method further comprises recovering L-glutamic acid from the cultured microorganism.
Embodiment 2-53. The method according to any one of Embodiments 2-49 to 2-52, wherein the production method further comprises recovering L-glutamic acid from medium in which the microorganism is cultured.
Embodiment 2-54. The method according to any one of Embodiments 2-49 to 2-53, wherein the culturing is performed with at least one source of carbon selected from the group consisting of carbohydrates, sugar alcohols, organic acids, amino acids, starch hydrolyzate, molasses, blackstrap molasses, rice winter, cassava, sugar cane offal and corn steep liquor.
Embodiment 2-55. The method according to any one of Embodiments 2-49 to 2-54, wherein the culturing is performed with at least one source of nitrogen selected from the group consisting of ammonia, ammonium sulfate, ammonium chloride, ammonium acetate, ammonium phosphate, ammonium carbonate, ammonium nitrate, glutamic acid, methionine, glutamine, peptone, NZ-amine, Beef extract, yeast extract, malt extract, com steep liquor, casein hydrolyzate, fish or degradation products thereof, and defatted soybean cake or degradation products thereof.
Embodiment 2-56. The method according to any one of Embodiments 2-49 to 2-55, wherein the culturing is performed with at least one selected from the group consisting of potassium phosphate, dipotassium phosphate, a sodium-containing salt corresponding thereto, sodium chloride, calcium chloride, iron chloride, magnesium sulfate, iron sulfate, manganese sulfate, and calcium carbonate.
Embodiment 2-57. A method of increasing L-glutamic acid production by a microorganism, comprising weakening an activity of a protein comprising an amino acid sequence having at least 85% sequence identity to the amino acid sequence of SEQ ID NO: 1 in the microorganism.
Embodiment 2-58. The method according to Embodiment 2-57, wherein the microorganism is the microorganism of any one of Embodiments 2-1 to 2-45.
Embodiment 2-59. A polynucleotide comprising a nucleic acid sequence encoding an amino acid sequence having at least 85% sequence identity to the amino acid sequence of SEQ ID NO: 1, wherein the polynucleotide does not occur in nature.
Embodiment 2-60. The polynucleotide according to Embodiment 2-59, wherein the amino acid sequence has at least 95% sequence identity to the amino acid sequence of SEQ ID NO: 1.
Embodiment 2-61. The polynucleotide according to Embodiment 2-59 or 60, wherein the amino acid molecule comprises the amino acid sequence of SEQ ID NO: 1.
Embodiment 2-62. The polynucleotide according to Embodiment 2-59 or 60, wherein the amino acid molecule does not comprise the amino acid sequence of SEQ ID NO: 1.
Embodiment 2-63. The polynucleotide according to any one of Embodiments 2-59 to 2-62, wherein the nucleic acid sequence comprises a start codon that does not naturally occur in encoding the amino acid sequence.
Embodiment 2-64. The polynucleotide according to any one of Embodiments 2-59 to 2-63, wherein the polynucleotide comprises a start codon, GTG.
Embodiment 2-65. The polynucleotide according to any one of Embodiment 2-59 to 2-63, wherein the polynucleotide comprises a start codon, TTG.
Embodiment 2-66. The polynucleotide according to any one of Embodiment 2-59 to 2-65, wherein the nucleic acid sequence comprises a Shine-Dalgarno sequence that does not naturally occur in encoding the amino acid sequence.
Embodiment 2-67. The polynucleotide according to any one of Embodiment 2-59 to 2-66, wherein the polynucleotide comprises a variant Shine-Dalgarno sequence, CAAGGCCGG.
Embodiment 2-68. The polynucleotide according to any one of Embodiment 2-59 to 2-66, wherein the polynucleotide comprises a variant Shine-Dalgarno sequence, CGTGACAGG.
Embodiment 2-69. The polynucleotide according to any one of Embodiment 2-59 to 2-66, wherein the polynucleotide comprises a variant Shine-Dalgarno sequence, CTTCGCTGG.
Embodiment 2-70. The polynucleotide according to any one of Embodiment 2-59 to 2-66, wherein the polynucleotide comprises a variant Shine-Dalgarno sequence, GTCGATTTC.
Embodiment 2-71. A vector comprising the polynucleotide of any one of Embodiment 2-59 to 2-70.
Embodiment 2-72. Use of composition product, process, or system characterized by one or more elements disclosed in the application.

1. A microorganism, in which an activity of a protein comprising a sequence having at least 85% sequence identity to the amino acid sequence of SEQ ID NO: 1 is weakened compared to an intrinsic activity of the microorganism.

2. A microorganism expressing a recombinant protein comprising an amino acid sequence having at least 85% sequence identity to the amino acid sequence of SEQ ID NO: 1.

3. The microorganism according to claim 1, wherein the microorganism of the genus Corynebacterium has L-glutamic acid producing ability.

4. The microorganism according to claim 1, wherein the protein comprises the amino acid sequence of SEQ ID NO: 1.

5. The microorganism according to claim 1, wherein the protein is encoded by a nucleotide molecule comprising the nucleotide sequence of SEQ ID NO: 2.

6. The microorganism according to claim 1, in which an expression control sequence for a gene encoding the protein is not an intrinsic sequence of the microorganism.

7. The microorganism according to claim 1, wherein the microorganism is Corynebacterium.

8. A method of producing L-glutamic acid, comprising culturing the microorganism of claim 1.

9. A method of increasing L-glutamic acid production by a microorganism, comprising weakening an activity of a protein comprising an amino acid sequence having at least 85% sequence identity to the amino acid sequence of SEQ ID NO: 1 in the microorganism of claim 1.

10. A polynucleotide comprising a nucleic acid sequence encoding an amino acid sequence having at least 85% sequence identity to the amino acid sequence of SEQ ID NO: 1, wherein the polynucleotide does not occur in nature.

11. Use of composition product, process, or system characterized by one or more elements disclosed in the application.
